(19) United States
(12) Patent Application Publication
(10) Pub. No.: US 2026/0069455 A1
(43) Pub. Date: Mar. 12, 2026

(54) DETERMINING THE PERPENDICULAR ALIGNMENT OF AN EYE

(71) Applicant: ALCON INC., Fribourg (CH)

(72) Inventor: Sissimos Lemonis, Ruckersdorf (DE)

(21) Appl. No.: 19/308,619

(22) Filed: Aug. 25, 2025

Related U.S. Application Data
(60) Provisional application No. 63/693,235, filed on Sep. 11, 2024.

(51) Int. Cl. A61F 9/008; A61B 17/00; G06T 7/00; G06T 7/521; G06T 7/73

(52) U.S. Cl. CPC A61F 9/008; G06T 7/0012; G06T 7/521; G06T 7/73; A61B 2017/00057; A61F 2009/00846; G06T 2207/30041

(57) ABSTRACT

In certain embodiments, a system for aligning an eye includes an illuminator, a camera system, and a computer. The illuminator directs an alignment pattern towards the eye at one or more positions. The alignment pattern is designed to indicate a perpendicular alignment of the eye with an ophthalmic system. The camera system generates an image of the alignment pattern reflected from the eye at each position of the one or more positions to yield one or more images. The computer performs an analysis of the one or more images to detect the perpendicular alignment of the eye with the ophthalmic system and determines the perpendicular alignment of the eye with the ophthalmic system in accordance with the analysis.

### FIELD

Embodiments of the present disclosure relate to determining the perpendicular alignment of an eye.

### BACKGROUND

During an ophthalmic procedure, the eye should be aligned with the ophthalmic device to properly perform the procedure. For example, in a laser ablation procedure, the cornea should be perpendicularly aligned with the center of ablation relative to the laser device to achieve the desired results. If the cornea is not perpendicularly aligned, the cornea is tilted and a portion of the cornea may be closer to the laser device than it should be, and another portion may be farther away. The resulting ablation may be deeper in the portion that is closer to the laser device and shallower in the portion that is farther away. Known techniques merely direct a pattern of light onto the cornea and then rely on the surgeon to use the reflection of the pattern to perpendicularly align the eye.

### SUMMARY

In certain embodiments, a system for aligning an eye includes an illuminator, a camera system, and a computer. The illuminator directs an alignment pattern towards the eye at one or more positions. The alignment pattern is designed to indicate a perpendicular alignment of the eye with an ophthalmic system. The camera system generates an image of the alignment pattern reflected from the eye at each position of the position(s) to yield one or more images. The computer performs an analysis of the image(s) to detect the perpendicular alignment of the eye with the ophthalmic system and determines the perpendicular alignment of the eye with the ophthalmic system in accordance with the analysis.

Embodiments may include one, two, more than one, or any combination of the following:
The computer provides an output representing the perpendicular alignment of the eye with the ophthalmic system.
The computer performs the analysis of the image(s) to detect the perpendicular alignment of the eye with the ophthalmic system by: detecting the alignment pattern in the image(s); and determining if at least a portion of the alignment pattern has a distortion indicating that there is a misalignment of the eye with the ophthalmic system. The computer may determine, according to the distortion, a direction of the misalignment of the eye. The computer may determine, according to a degree of the distortion, an amount of the misalignment of the eye. The computer may determine a direction of the misalignment of the eye and provide an output indicating the direction of the misalignment of the eye. The computer may determine an amount of the misalignment of the eye and provide an output indicating the amount of the misalignment of the eye.
The position(s) of the alignment pattern comprising a first position of the alignment pattern and a second position of the alignment pattern, where the second position of the alignment pattern is rotated about a central point of the eye away from the first position of the alignment pattern. The second position of the alignment pattern may be rotated about the central point of the eye away from the first position of the alignment pattern by an angle of 25 to 95 degrees.
The illuminator directs the alignment pattern towards the eye at the position(s) by directing a first alignment pattern towards the eye at one or more first positions and directing a second alignment pattern towards the eye at one or more second positions. The camera system may generate the image of the alignment pattern reflected from the eye at the each position of the position(s) to yield the image(s) by generating a first image of the first alignment pattern reflected from the eye at the each first position of the first position(s) to yield one or more first images and generating a second image of the second alignment pattern reflected from the eye at the each second position of the second position(s) to yield one or more second images. The computer may perform the analysis of the image(s) to detect the perpendicular alignment of the eye with the ophthalmic system by performing a first analysis of one or more first images of the first alignment pattern reflected from the eye to detect the perpendicular alignment of the eye with the ophthalmic system and performing a second analysis of one or more second images of the second alignment pattern reflected from the eye to detect the perpendicular alignment of the eye with the ophthalmic system.
The computer determines a central point of the eye with which the ophthalmic system is to be perpendicularly aligned. The computer may determine the central point of the eye with which the ophthalmic system is to be perpendicularly aligned by accessing a treatment plan indicating the central point. The computer may determine the central point of the eye with which the ophthalmic system is to be perpendicularly aligned by receiving a user input indicating the central point. The illuminator may direct the alignment pattern towards the eye such that the alignment pattern is centered about the central point.
The illuminator ceases directing the alignment pattern towards the eye when an eye-tracker is directing an eye-tracking light towards the eye.
The computer accesses a description of a shape of an anterior surface of the eye and designs the alignment pattern according to the description of the shape of the anterior surface of the eye.
The computer accesses a description of a shape of an anterior surface of the eye and selects at least one position of the one or more positions according to the description of the shape of the anterior surface of the eye.
The illuminator directs a test pattern towards the eye at one or more test positions. The camera system generates a test image of the test pattern reflected from the eye at each test position of the one or more test positions to yield one or more test images. The computer performs a test analysis of the test image(s) to yield a description of a shape of an anterior surface of the eye. The computer may design the alignment pattern according to the description of the shape of the anterior surface of the eye. The computer may select at least one position according to the description of the shape of the anterior surface of the eye.
The computer determines that a perpendicular misalignment exceeds a safe procedure limit and pauses an operation of a laser of the ophthalmic system.
The computer determines that a perpendicular misalignment exceeds a safe procedure limit and provides an output indicating that the perpendicular misalignment exceeds the safe procedure limit.
The computer determines that a perpendicular misalignment is within a caution zone and provides an output indicating that the perpendicular misalignment is within the caution zone.

### BRIEF DESCRIPTION OF THE DRAWINGS

FIG. 1 illustrates an example system for perpendicularly aligning an eye, according to at least one embodiment described in the present disclosure;

FIG. 2 illustrates an example of the perpendicular alignment of a cornea, according to at least one embodiment described in the present disclosure;

FIG. 3 illustrates an example computer system, according to at least one embodiment described in the present disclosure;

FIGS. 4A and 4B illustrates examples of an alignment pattern reflected from an eye, according to at least one embodiment described in the present disclosure;

FIGS. 5A to 5H illustrate examples of an alignment pattern directed at different positions of an eye, according to at least one embodiment described in the present disclosure;

FIGS. 6A to 6D illustrates examples of patterns that can be directed towards an eye, according to at least one embodiment described in the present disclosure; and

FIG. 7 illustrates an example of a method of determining the perpendicular alignment of an eye, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

During an ophthalmic procedure, the eye should be aligned with the ophthalmic device to properly perform the procedure. For example, in a laser ablation procedure, it is beneficial to align the cornea perpendicularly relative to the laser device (e.g., relative to the optical axis and/or the treatment plane of the laser device), in order to achieve the planned ablation results. If the cornea is not perpendicularly aligned, the cornea is tilted and a portion of the cornea may be closer to the laser device than expected, and another portion may be farther away than expected. Ablation patterns are typically designed for perpendicularly aligned corneas. Accordingly, if the cornea is tilted, the resulting ablation may be deeper than planned in the portion that is closer to the laser device and shallower than planned in the portion that is farther away. These effects tend to be greater towards the periphery of the cornea.

Known techniques merely direct a pattern of light onto the cornea and then rely on the surgeon to use the reflection of the pattern to perpendicularly align the eye. These known techniques rely on the surgeon's subjective determinations and do not provide objective perpendicular alignment. In addition, the known techniques do not indicate any change in the alignment, which may be caused by the patient's movement. Furthermore, the known techniques cannot provide perpendicular alignment for ablation profiles that are centered at a point different from the pupil center, which is typically used as the centration point for the optical axis of the laser.

The present disclosure relates to determining the perpendicular alignment of the eye with an ophthalmic device. According to embodiments of the present disclosure, an illuminator directs an alignment pattern towards the eye. The alignment pattern is designed to indicate perpendicular alignment. A camera generates images of the alignment pattern on the eye. A computer system analyzes the images to determine the perpendicular alignment of the eye.

Certain embodiments of the present disclosure may provide improvements over previous iterations of determining perpendicular alignment of the eye. For example, certain embodiments may automatically determine the perpendicular alignment without relying on a user's subjective determination. As another example, certain embodiments may provide guidance to the user for correcting the misalignment. As another example, certain embodiments may detect a perpendicular misalignment during a procedure and may provide a notification of the perpendicular misalignment. As another example, certain embodiments may allow a user to select the center point about which the eye is to be perpendicularly aligned, e.g., the corneal apex, the corneal vertex, the pupil center, or other suitable central point. As another example, certain embodiments may provide any suitable output in response to determining the perpendicular alignment, e.g., may describe the direction and/or amount of misalignment, may display a notification of misalignment, and/or may pause a laser procedure if there is misalignment.

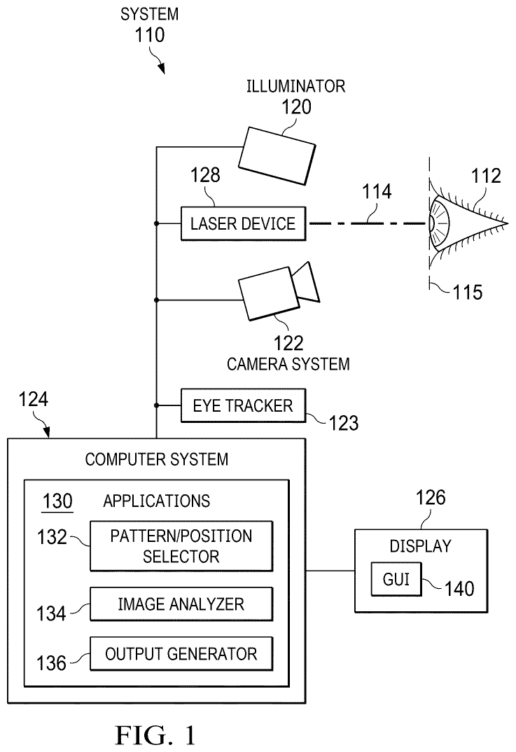

FIG. 1 illustrates an example system 110 for perpendicularly aligning an eye 112, according to at least one embodiment described in the present disclosure. In the illustrated example, the system 110 includes an illuminator 120, a camera system 122, an eye-tracker 123, a computer system 124, a display 126, and a laser device 128 with an optical axis 114 and a treatment plane 115, which may be coupled as shown. The computer system 124 includes software applications 130, such as a pattern/position selector 132, an image analyzer 134, and an output generator 136.

In an example of operation, the illuminator 120 directs an alignment pattern towards the eye 112 at one or more positions (e.g., locations and/or orientations). The alignment pattern is designed to indicate a perpendicular alignment of the eye 112 with the system 110 (e.g., with the optical axis 114 and/or the treatment plane 115 of the system 110) relative to a central point (e.g., the corneal apex, the corneal vertex, the pupil center, or other suitable central point). The camera system 122 generates an image of the alignment pattern on the eye at each position of the one or more positions to yield one or more images. The computer system 124 performs an analysis of the one or more images to detect the perpendicular alignment of the eye 112 with the system 110. The computer system 124 determines the perpendicular alignment of the eye 112 with the system 110 in accordance with the analysis.

The embodiments may use any suitable geometric references. For ease of explanation, embodiments may be described using the following example geometric references. An example xyz-coordinate system may be relative to the coordinate system of the system 110, where the z-axis is aligned with the optical axis 114 of the system 110. The xy-plane is orthogonal to the z-axis and may have any suitable x-axis (e.g., a horizontal axis of the eye) and any suitable y-axis (e.g., a vertical axis of the eye). The optical axis 114 may be any suitable optical axis of the system 110, such as the optical axis of the laser device 128 or the optical axis of the eye-tracker 123. The eye 112 has a corneal vertex, a corneal apex, and a pupil center. A central point of the eye 112 may refer to the corneal vertex, the corneal apex, the pupil center, or other suitable point or may refer to a point defined relative to one of or any combination of the corneal vertex, the corneal apex, the pupil center, or other suitable point, e.g., a point on a line between the corneal vertex and the pupil. The pupil center may be determined in any suitable manner, e.g., the pupil center may be a Line Of Sight (LOS) pupil center determined when the patient looks at a fixation light.

Turning to examples of the components of the system 110, the illuminator 120 illuminates and directs light in a pattern towards a target, such as the eye 112. Any suitable wavelength of light may be used, e.g., visible light and/or invisible light (such as infrared light). Any suitable pattern may be used. For example, the pattern may comprise one or more of any suitable graphical elements, such as lines (e.g., solid, dotted, and/or dashed), shapes (e.g., ellipses, circles, polygons, and/or squares), and/or patterns (e.g., an array (such as a rectangular or circular array) of lines and/or shapes). The lines may have any suitable length, thickness, curvature, separation, and/or pattern (e.g., dashed, dotted, or any combination of dashes and dots). For ease of explanation, a pattern may have a reference point, such as a pattern center point (e.g., a centroid), and a pattern axis that goes through the reference point.

The pattern may be, e.g., an alignment pattern and/or a test pattern. In certain embodiments, an alignment pattern is designed to indicate a perpendicular alignment of the eye with an ophthalmic system (e.g., with the optical axis 114 and/or treatment plane 115 of the system 110) relative to a central point. In the embodiments, an image of the alignment pattern reflected from the eye 112 is obtained. The alignment pattern may have one appearance if the eye 112 is perpendicularly aligned and a different appearance if the eye 112 is not perpendicularly aligned. For example, the pattern may have a change in appearance, e.g., distortion, that appears in the alignment if the eye 112 is not perpendicularly aligned. The change in appearance may be proportional to the degree of misalignment, e.g., a greater degree of misalignment yields a greater change in appearance. For example, the pattern may have one appearance (e.g., a symmetrical appearance) when reflected from a perpendicularly aligned eye, but the pattern may have a different appearance (e.g., an asymmetrical appearance) when reflected from a perpendicularly misaligned eye. Examples of alignment patterns are described herein.

In certain embodiments, a test pattern is designed to describe a feature of the eye, such as the shape of the anterior corneal surface and/or sclera of the eye. In some cases, the shape of the anterior corneal surface and/or sclera of the eye may be used to select an alignment pattern and/or select positions towards which an alignment pattern is to be directed. The test pattern may be, e.g., a pattern used to determine the topography of an eye, e.g., a Placido disk pattern. Examples of test patterns are described herein.

The illuminator 120 may direct a pattern towards the eye 112 at any suitable position, where the position of a pattern may be described as, e.g., the location of the pattern on the cornea and/or the orientation of the pattern. The pattern may be directed towards any suitable location of the cornea of the eye 112. For example, the pattern may be centered about any suitable point, e.g., a central point of the eye 112. In addition, the pattern may be directed at any suitable orientation relative to the eye 112. For example, the pattern may be directed such that the pattern axis is at any suitable angle relative to the y-axis. Moreover, the pattern may be moved from one position (e.g., one location and/or orientation) to another position (e.g., another location and/or orientation), as described herein.

One or more patterns may be directed towards the eye 112 one or more times in any suitable manner. For example, a pattern may be directed towards the eye 112 multiple times. Each time, the position of the pattern (e.g., the location on the cornea and/or the orientation of the pattern) may be the same as or may be different from a previous position (e.g., a previous location and/or a previous orientation). As another example, two or more patterns may be directed towards the eye 112, e.g., a first alignment pattern may be directed towards the eye at one or more first positions and a second alignment pattern may be directed towards the eye at one or more second positions. The patterns may be used in any suitable order, e.g., round-robin order, predefined order, and/or random order. The position of the pattern may be the same as or may be different from a previous position that was used.

In certain embodiments, the illuminator 120 may direct the pattern such that the pattern is centered about a central point of the eye 112, e.g., the centroid of the pattern may be centered about the central point of the eye 112. The illuminator 120 may then rotate the pattern about the centroid to different orientations. The second position may be rotated about the optical axis 114 of the ophthalmic system away from the first position by an angle in the range of, e.g., 0 to 90 degrees, such as 0 to 10, 10 to 15, 15 to 30, 30 to 45, and/or 45 to 90 degrees. The illuminator 120 may rotate the pattern in any suitable combination of angles, e.g., the pattern may be directed at an angle of 0 degrees relative to the y-axis and then at an angle of 90 degrees relative to the y-axis, or at any other suitable combination of one, two, or more angles.

The illuminator 120 may comprise any suitable components and operate in any suitable manner to direct patterns toward the eye 112. In certain embodiments, the illuminator 120 may comprise a system of one or more light source(s) arranged in a manner that yields the pattern. For example, the illuminator 120 may comprise a stripe illuminator (e.g., a prism illuminator) that yields a striped pattern. The illuminator 120 may move the light source system to direct the pattern to a different position (e.g., a different location on the cornea and/or a different orientation of the pattern). For example, the illuminator 120 may rotate a stripe illuminator about the optical axis 114 to rotate the striped pattern. In certain embodiments, the illuminator 120 may comprise a system (e.g., an array) of light sources that can be turned on or off in a manner that yields the pattern. The illuminator 120 may turn on and/or off the light sources to move the pattern to a different position. In certain embodiments, the illuminator 120 may comprise one or more optical devices that direct light from a light source in a manner that yields the pattern. For example, a prism (such as a rotational prism) may direct the pattern to different orientations. As another example, a scanner may scan the light to yield the pattern. In certain embodiments, the illuminator 120 may include light sources that provide light of different wavelengths. For example, one source provides light of a visible wavelength, and a different source provides light of a non-visible wavelength. In the example, if the source that provides the non-visible light fails, the surgeon may still use the visible light to align the eye.

In certain embodiments, the illuminator 120 may direct a pattern towards the eye in a manner that is coordinated with other operations of the system 110. In some embodiments, the illuminator 120 may illuminate the pattern such that system 110 can monitor the perpendicular alignment of the eye 112 and track the location of the eye 112. For example, the illuminator 120 illuminates the pattern when the eye-tracker 123 is not tracking the eye 112 and does not illuminate the pattern when the eye-tracker 123 is tracking the eye 112. In the example, the computer system 124 may instruct the illuminator 120 to cease the illumination of the alignment pattern when the eye-tracker 123 is directing eye-tracking light towards the eye.

In some embodiments, the illuminator 120 may illuminate the pattern such that system 110 can monitor the perpendicular alignment of the eye 112 and operate the laser device 128. For example, the illuminator 120 illuminates the pattern when the laser device 128 is not emitting a laser beam and does not illuminate the pattern when the laser device 128 is emitting a laser beam. In the example, the computer system 124 may instruct the illuminator 120 to cease the illumination of the alignment pattern when the laser device 128 is emitting a laser beam.

The camera system 122 may include one or more cameras or other optical sensors that can generate an image of the eye 112. In general, a digital camera or other optical sensor detects light from an object and generates a signal in response to the light. The signal carries digital image data that can be used to generate the digital image of the object. A camera may have any suitable frame rate, e.g., 50 to 2000 frames per second, such as 50 to 200, 200 to 500, 500 to 1000, 1000 to 1500, and/or 1500 to 2000 frames per second. Examples of digital cameras include a charged-coupled device (CCD) camera, a video camera, a complementary metal-oxide semiconductor (CMOS) sensor (e.g., active-pixel sensor (APS)), a line sensor, and an optical coherence tomography (OCT) camera.

In certain embodiments, the camera system 122 may include an alignment camera that captures images of a pattern reflected from the eye 112, and/or an eye-tracking camera that captures images of the eye 112 to track the movement of the eye 112, and/or a multi-purpose camera that captures images of a pattern reflected from the eye 112 and/or captures images of the eye 112 to track the movement of the eye 112. For example, an alignment procedure may use 50 to 500 frames per second (such as 50 to 200, 200 to 400, and/or 400 to 500 frames per second), and an eye-tracking procedure may use 50 to 1500 frames per second. A camera that can take 600 to 2000 frames per second can obtain images for eye-tracking and images for perpendicular alignment. In an example, a camera obtains, e.g., 1000 images per second. The images may alternatingly be used from the eye-tracking procedure and the alignment procedure such that the eye-tracking procedure uses, e.g., 500 images per second and the alignment procedure uses 500, e.g., images per second. In another example, one of more of the images may be used for more than one procedure, e.g., any combination of the eye-tracking procedure, the alignment procedure, and/or the laser beam emitting procedure.

The computer system 124 controls components and operations of the system 110. For example, the computer system 124 performs an analysis of one or more images to detect the perpendicular alignment of the eye 112 relative to the system 110. Examples of the computer system 124 are described herein. The computer system 124 includes software applications 130, such as the pattern/position selector 132, the image analyzer 134, and the output generator 136.

The pattern/position selector 132 selects an alignment pattern to be used and/or the position (e.g., the location on the cornea and/or the orientation of the pattern) at which the alignment pattern is to be directed. The pattern/position selector 132 may select an alignment pattern in any suitable manner. For example, the pattern/position selector 132 may use a default alignment pattern and/or may receive a user selection of the alignment pattern. The pattern/position selector 132 may select the positions at which the alignment pattern is to be directed in any suitable manner. The pattern/position selector 132 may select the positions at which the alignment pattern is to be directed in any suitable manner. For example, the pattern/position selector 132 may use a default position, a position corresponding to the alignment pattern, and/or a user selection of the position.

In certain embodiments, the pattern/position selector 132 may try different candidate settings, e.g., different alignment patterns, different positions, and/or different combinations of different alignment patterns at different positions for an eye 112 that is aligned, an eye 112 that is not aligned, or an eye 112 that is aligned and then not aligned. The pattern/position selector 132 may identify the candidate settings that yield the most stable results (e.g., results that consistently indicate that the aligned eye 112 is aligned and/or the not aligned eye 112 is not aligned) and select the identified candidate settings.

In certain embodiments, the pattern/position selector 132 may design or select an alignment pattern and/or position according to the shape of the anterior surface of the eye 112, which may be determined in any suitable manner. For example, the pattern/position selector 132 may access a description of the shape of the anterior surface. As another example, the illuminator 120 may direct a test pattern towards the eye at one or more test position(s), the camera system 122 may generate a test image of the test pattern at the position(s) to yield one or more test image(s), and the computer system 124 may perform a test analysis of the test image(s) to determine the shape of the anterior surface.

In the embodiments, the pattern/position selector 132 may design or select an alignment pattern and/or position according to the shape of the anterior surface of the eye 112. For example, an astigmatic eye typically has a steeper meridian and a flatter meridian. Rotation about an axis defined by the flatter meridian yields smaller changes in a reflected light pattern than rotation about an axis defined by the steeper meridian. In the example, the pattern/position selector 132 may design or select an alignment pattern and/or position that can detect the smaller changes in rotation about the axis defined by the flatter meridian. For example, a pattern with one or more region(s) that exhibit greater distortion along the steeper meridian may be selected, or the pattern may be positioned such that the region(s) are placed at or near the steeper meridian.

The image analyzer 134 detects and analyzes an image of an alignment pattern reflected from an eye 112 to determine the perpendicular alignment of the eye 112. An alignment pattern is designed to indicate a perpendicular alignment of the eye relative to the system 110, e.g., the optical axis 114 and/or treatment plane 115 of the system 110. The alignment pattern may have one appearance if the eye 112 is perpendicularly aligned and a different appearance if the eye 112 is not perpendicularly aligned. The alignment pattern may also have a reference point (e.g., a center point) and/or a pattern axis. The reference point may be designed to coincide with a reference point of the eye 112 (e.g., a central point of the eye 112) when the eye 112 is aligned in an xy-plane. Accordingly, the image analyzer 134 may also determine the xy-alignment of the eye 112.

In certain embodiments, even when the eye 112 is centered at a first central point of the eye 112 (e.g., the pupil center) when the images are obtained, the image analyzer 134 can determine the perpendicular alignment of the eye 112 relative to a second central point (e.g., the pupil center). In the embodiments, the image analyzer 134 may apply a translation function to the coordinates of the image to translate the coordinates from being centered at the first central point (e.g., the pupil center) to being centered at the second central point (e.g., the pupil center).

In certain embodiments, the image analyzer 134 analyzes an image of a test pattern reflected from the eye 112 to determine the topography of the eye 112. The test pattern may be any suitable pattern used to determine the topography of an eye, e.g., a Placido disk pattern. The image analyzer 134 may analyze the test pattern reflected from the eye 112 to determine the shape of the anterior surface of the eye. For example, the image analyzer 134 may detect distortions of the test pattern that may indicate a deviation of the surface from a spherical curvature.

The output generator 136 may generate any suitable output, e.g., an output presented via the GUI 140, the display 126, and/or other interface described herein. For example, the output generator 136 outputs information representing the perpendicular alignment of the eye 112, such as a notification indicating the direction and/or amount of the misalignment of the eye 112. The direction of the misalignment may be expressed in any suitable manner, e.g., relative to the xy-plane with the optical axis 114 intersecting (x, y)=(0, 0). The amount of the misalignment may be expressed in any suitable manner, e.g., as an angular measurement relative to the optical axis 114.

As another example, the output generator 136 determines that the misalignment is within a caution zone and provides a warning indicating that the misalignment is within the caution zone. The caution zone may be expressed in any suitable manner, e.g., as a range of angles relative to the optical axis 114, such as within 0 to 1, 1 to 2, 2 to 3, 3 to 4, and/or 4 to 5 degrees of the aligned position at the optical axis 114. As another example, the output generator 136 determines that the misalignment exceeds a safe procedure limit and then provides a warning indicating that the misalignment exceeds the safe procedure limit and/or pauses an operation of a laser of the ophthalmic system. A safe procedure limit may be expressed in any suitable manner, e.g., as an angle relative to the optical axis 114, such as within 5 degrees, within 2 degrees, or within 1 degree of the aligned position at the optical axis 114.

In certain embodiments, the computer system 124 may coordinate the operation of the illuminator 120 with one or more other components of the system 110. For example, the computer system 124 may instruct the illuminator 120 to illuminate the pattern when the eye-tracker 123 is not tracking the eye 112 and/or to not illuminate the pattern when the eye-tracker 123 is tracking the eye 112. As another example, the computer system 124 may instruct the eye-tracker 123 to track the eye when 112 the illuminator 120 is not illuminating the pattern and/or to not track the eye when 112 the illuminator 120 is illuminating the pattern. As another example, the computer system 124 may instruct the illuminator 120 to illuminate the pattern when the laser device 128 is not emitting a laser beam and/or to not illuminate the pattern when the laser device 128 is emitting a laser beam. As another example, the computer system 124 may instruct the laser device 128 to emit a laser beam when 112 the illuminator 120 is not illuminating the pattern and/or to not emit a laser beam when 112 the illuminator 120 is illuminating the pattern. As another example, to reduce interference, the computer system 124 may detect a brighter area of an image of the eye, where the brighter area represents a laser pulse, and then blend the brighter area with the corresponding area from an image of a pattern on the eye.

The laser device 128 may be used to treat an eye. The laser device 128 has an optical axis 114 and is designed to provide treatment at a treatment plane 115. In certain embodiments, the laser device 128 directs a laser beam towards the treatment plane 115 to, e.g., perform a surgical procedure. Examples of the laser device 128 include excimer and femtosecond lasers. The laser beam may have any suitable pulse duration, such as in the order of nanoseconds, picoseconds, femtoseconds, or attoseconds. The laser beam may have any suitable wavelength, such as in the range of 150 nanometers (nm) to 20 micrometers (μm). Examples of ranges include the ultraviolet (e.g., in the range of 180 to 400 nm, such as 190 to 195 nm or 345 to 355 nm), visible, or infrared wavelength (e.g., in the range of 1050 to 1250 or 1250 to 1500 nm). The laser beam may process material in any suitable manner, e.g., ablate, incise, or photo-disrupt the material.

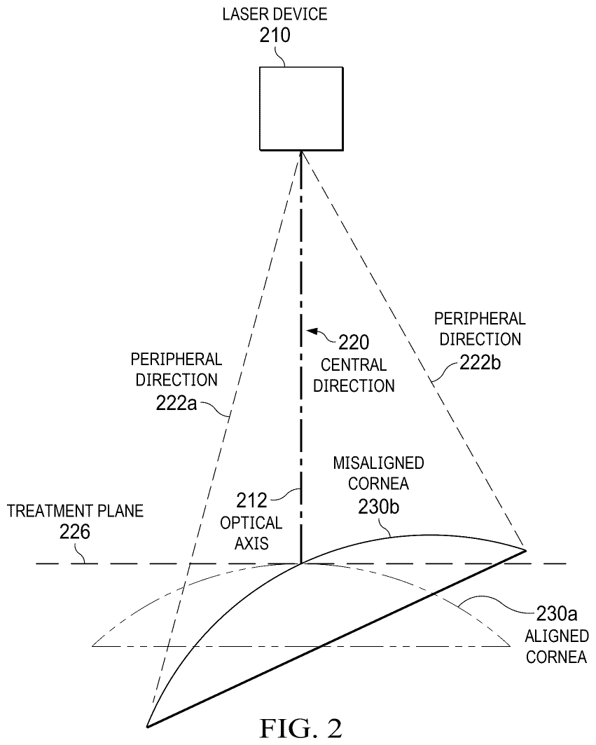

FIG. 2 illustrates an example of the perpendicular alignment of a cornea 230 (230a and/or 230b), according to at least one embodiment described in the present disclosure. In the example, a laser device 210 may be used to treat an eye with a cornea 230. The laser device 210 has an optical axis 212 and provides treatment at a treatment plane 226 according to a treatment plan. The laser device 210 can direct a beam in different directions towards the cornea 230, such as a central direction 220 aligned with the optical axis 212 and/or peripheral directions 222 (222a and/or 222b) apart from the optical axis 212 towards and/or at the peripheral of the scope of the laser device 210.

The aligned cornea 230a is centered and perpendicularly aligned with the optical axis 212. Although the aligned cornea 230a is aligned, the periphery of the aligned cornea 230a is below the treatment plane 226. As a consequence, the periphery receives a weaker beam, due to laser efficiency affected by the angle of incidence and pulse projection. In certain cases, a pre-compensation matrix may compensate for this weakness by, e.g., adding pulses (e.g., up to 35% additional pulses) to the periphery such that the aligned cornea 230a receives the proper amount of laser radiation.

The misaligned cornea 230b is not perpendicularly aligned with the optical axis 212. Accordingly, the portion of the misaligned cornea 230b proximate to peripheral direction 222a is below the treatment plane 226 and is ablated less than according to the treatment plan. The portion of the misaligned cornea 230b proximate to peripheral direction 222b is above the treatment plane 226 and is ablated more than according to the treatment plan. The pre-compensation matrix is designed to compensate for the calculated beam efficiency for the aligned cornea 230a, not the misaligned cornea 230b. As a consequence, the misaligned cornea 230b receives the incorrect treatment. Moreover, since the pupil is approximately 3.5 millimeters (mm) away from the corneal surface, the misalignment may cause decentration.

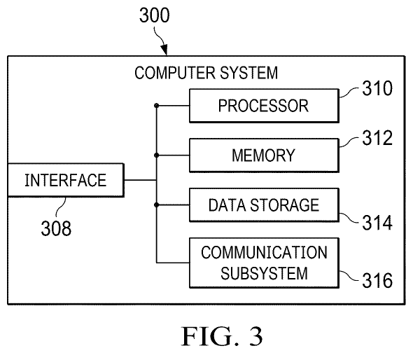

FIG. 3 illustrates an example computer system 300, according to at least one embodiment described in the present disclosure. The computer system 300 may include an interface 308, a processor 310, a memory 312, a data storage 314, and/or a communication subsystem 316, any or all of which may be communicatively coupled. Any or all of the computer system 300 may be implemented as computer hardware and/or software. Any or all of the computer systems described herein may be implemented as a computer system consistent with the computer system 300.

In the example, the interface 308 may receive input to the computer system 300 and/or send output from the computer system 300, and may be used to exchange information between, e.g., software, hardware, one or more peripheral devices, one or more users, and/or any suitable combinations of any of the preceding. A user interface is a type of interface that a user can utilize to communicate with (e.g., send input to and/or receive output from) the computer system 300. Examples of user interfaces include displays, Graphical User Interfaces (GUIs), touchscreens, foot pedals, keyboards, computer mouses (or mice), gesture sensors, microphones, and speakers.

Generally, the processor 310 may include any suitable special-purpose or general-purpose computer, computer entity, or processing device including various computer hardware or software modules and may be configured to execute instructions stored on any applicable computer-readable storage media. For example, the processor 310 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 3, the processor 310 may include any number of processors distributed across any number of network or physical locations that are configured to perform individually or collectively any number of operations described in the present disclosure.

The processor 310 may perform any suitable operations. In some embodiments, the processor 310 may interpret and/or execute program instructions and/or process data stored in the memory 312, the data storage 314, or the memory 312 and the data storage 314. In some embodiments, the processor 310 may fetch program instructions from the data storage 314 and load the program instructions into the memory 312. After the program instructions are loaded into the memory 312, the processor 310 may execute the program instructions, such as instructions to perform any of the methods disclosed herein, respectively.

The memory 312 and the data storage 314 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 310.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 310 to perform a certain operation or group of operations.

The communication subsystem 316 may include any component, device, system, or combination thereof that is configured to transmit, receive, and/or otherwise exchange information over a network in order to communicate with any suitable entity, such as with other devices at other locations or at the same location or even within the same system. The communication subsystem 316 may provide for communication among the devices described in the present disclosure, communication networks, computing devices, and other systems. For example, the communication subsystem 316 may allow the system 300 to communicate with other systems, such as other computing devices and/or networks. In some embodiments, the communication subsystem 316 may include a modem, a network card (wireless or wired), an optical communication device, an infrared communication device, a wireless communication device (such as an antenna), and/or chipset. Examples of communication subsystem 316 include a Bluetooth device, an 802.6 device (e.g., that can communicate with a Metropolitan Area Network (MAN)), a WiFi device, a WiMax device, cellular communication facilities, and/or the like.

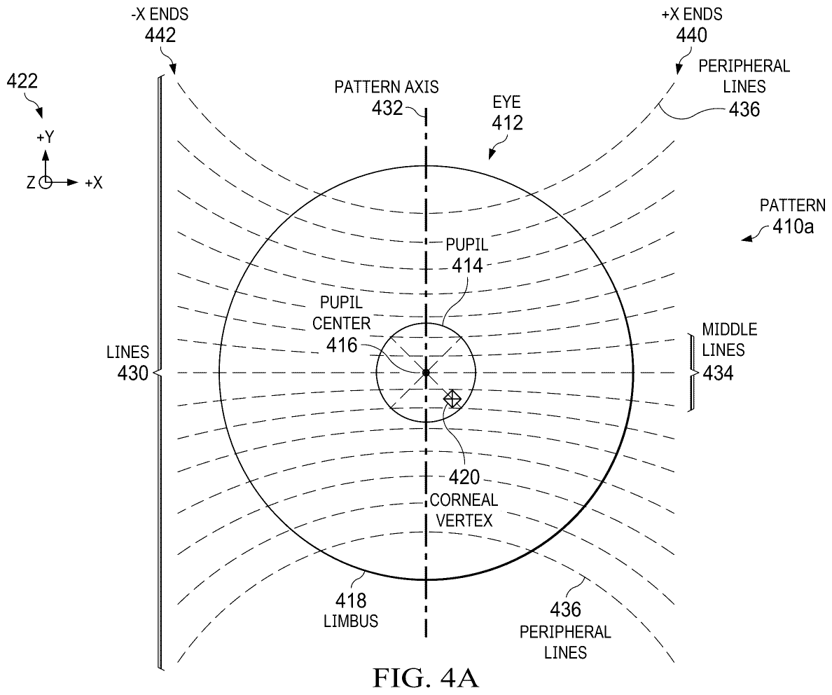

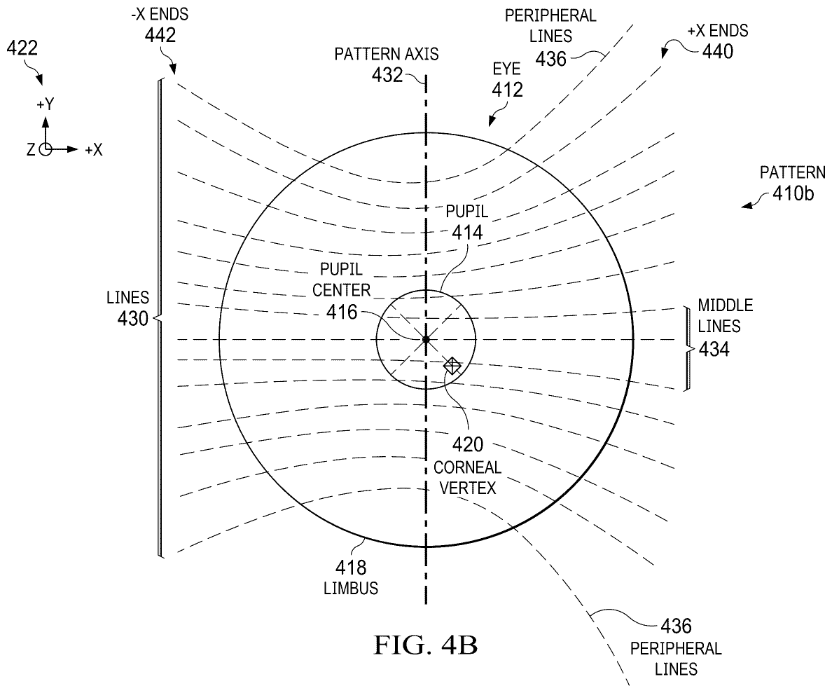

FIGS. 4A and 4B illustrates examples of an alignment pattern 410 (410a and/or 410b) reflected from an eye 412, according to at least one embodiment described in the present disclosure. The eye 412 includes a pupil 414 (with a pupil center 416), a limbus 418, and a corneal vertex 420. For ease of explanation, a coordinate system 422 (which may be a coordinate system of an ophthalmic system) is shown. The illuminator 120 provides an initial alignment pattern, which is the pattern before being reflected by the eye 412. The initial alignment pattern may comprise any suitable number of lines arranged in any suitable manner that, when reflected by the eye 412, can indicate the perpendicular alignment of the eye 412. The lines of the initial alignment pattern may have any suitable length, thickness, curvature (e.g., straight or curved), and/or line pattern, e.g., dashed, dotted, or any combination of dashes and dots. For example, the lines may be selected such that the pattern, when reflected by the eye 412, covers the cornea and may reach the sclera. The lines of the initial alignment pattern may be any suitable distance apart from each other and may or may not be evenly spaced from each other.

The illustrated alignment pattern 410 is the pattern reflected from the eye 412. In the example, the reflected alignment pattern 410 has reflected lines 430 with +x ends 440 in the +x direction and −x ends 442 in the −x direction. The lines 430 include one or more middle lines 434 located near or at the center of the alignment pattern 410 and one or more peripheral lines 436 located near or at the periphery of the alignment pattern 410. The middle lines 434 and the peripheral lines 436 may have any suitable curvature. Since the eye 412 that reflects the pattern is a generally spherical shape, the middle lines 434 are straighter (have less curvature) than the peripheral lines 436. The alignment pattern 410 may have a pattern axis 432 that one or more lines 430 may intersect, e.g., the middle of the lines 430 intersect the pattern axis 432.

The alignment pattern 410 may be centrally aligned with any suitable point of the eye 412, e.g., the point of the eye 412 at which a laser device centrally aligns a treatment pattern (which may be determined by the ophthalmic system or may be input by the user). For example, the alignment pattern 410 may be aligned with a central point of the eye 112, such as the pupil center 416 or the corneal vertex 420. In the example, the alignment pattern 410 is centrally aligned with the pupil center 416.

FIG. 4A shows the alignment pattern 410a reflected from the eye 412 when the eye is perpendicularly aligned, and FIG. 4B shows the alignment pattern 410b reflected from the eye 412 when the eye 412 is not perpendicularly aligned, e.g., the eye 412 is tilted in the +x-direction. In the example, the alignment pattern 410a has one appearance (e.g., a symmetrical appearance) when reflected from the perpendicularly aligned eye 412. The lines 430 of the alignment pattern 410a are substantially reflective symmetric about the pattern axis 432. The alignment pattern 410b has a different appearance (e.g., an asymmetrical appearance) when reflected from the perpendicularly misaligned eye 412. The alignment pattern 410a is distorted in that the lines 430 of the alignment pattern 410a are not symmetric about the pattern axis 432. For example, distance between the +x ends 440 of the lines 430 has increased, and the distance between the −x ends 442 of the lines 430 of the alignment pattern 410b has decreased.

In certain embodiments, the positioning relative to the coordinate system 422 indicates the direction of the perpendicular misalignment. In the example, the pattern axis 432 of the alignment pattern 410 is substantially parallel to the y-axis of the coordinate system 422. The distance between the +x ends 440 of the lines 430 is larger, indicating that the +x portion of the eye 412 is farther away, and the distance between the −x ends 442 of the lines 430 of the alignment pattern 410b is smaller, indicating that the-x portion of the eye 412 is closer. Accordingly, the alignment pattern 410b indicates that the eye 412 is tilting in the +x direction.

An alignment pattern may be used to determine the amount of perpendicular misalignment. In the example, the lines 430 are substantially reflective symmetric about the pattern axis 432 when reflected from a perpendicularly aligned eye 412. That is, the distance between the +x ends 440 of adjacent lines 430 generally match the distance between the-x ends 442 of the lines 430. Accordingly, a difference in the distances can indicate perpendicular misalignment and generally a greater difference indicates a greater perpendicular misalignment. The mathematical relationship between the differences and the amount of perpendicular misalignment may be determined by testing the alignment pattern 410 with an eye (or an eye model) at different perpendicular alignments and/or calculating the mathematical relationship according to geometrical equations. A computer may store and use the mathematical relationship to determine the amount of perpendicular misalignment from an image of the alignment pattern 410 reflected from an eye.

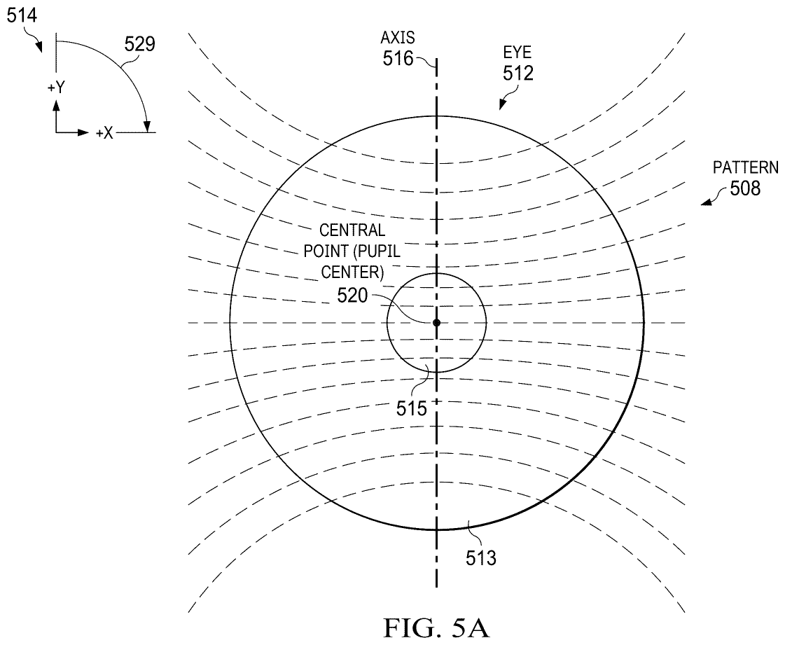

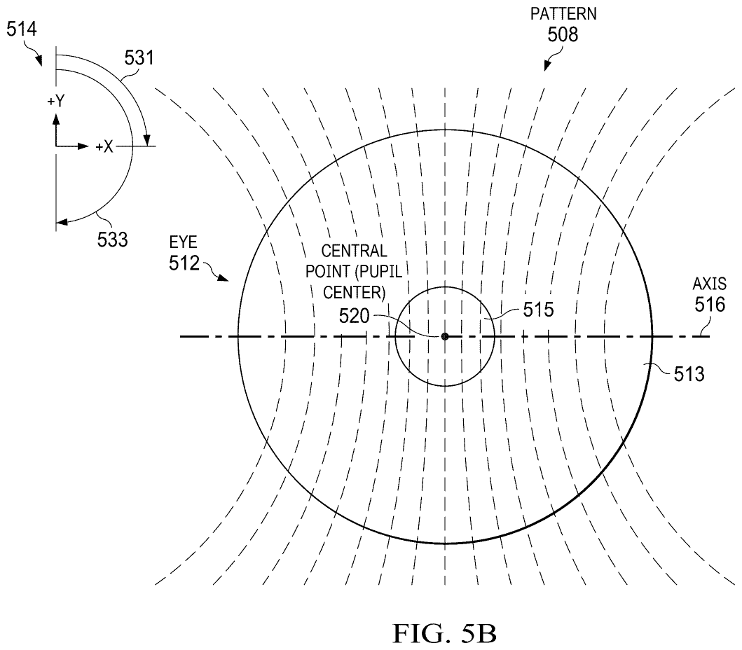

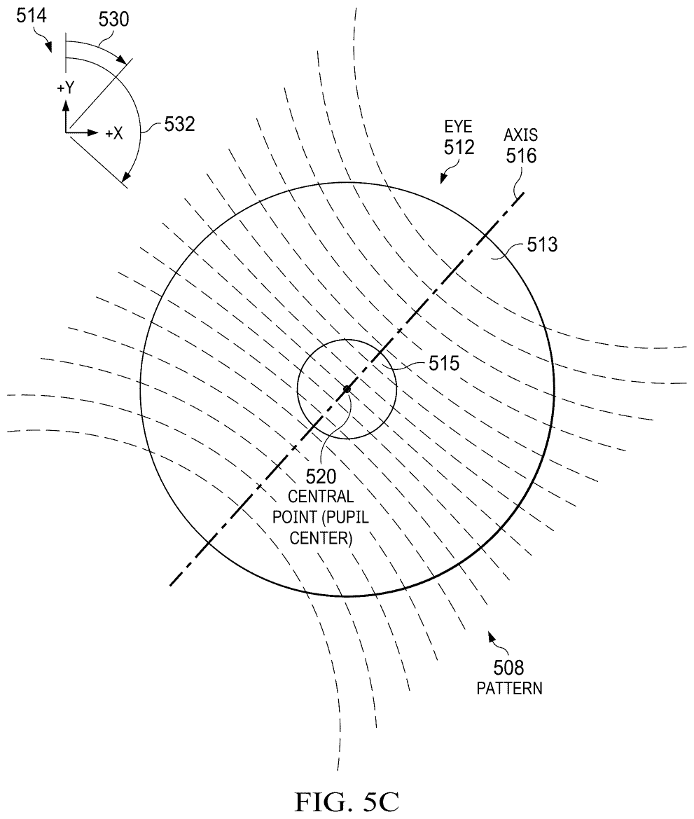

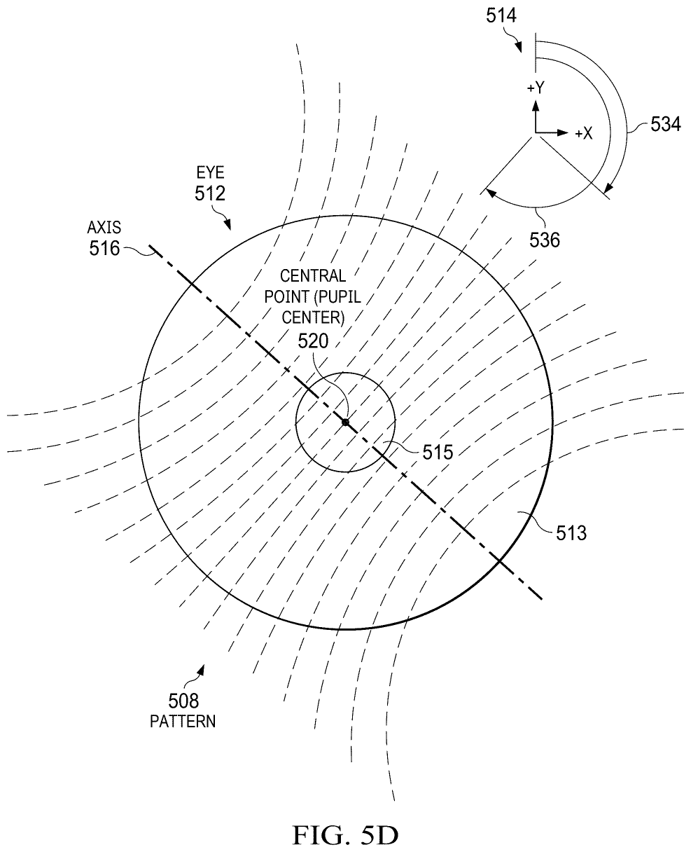

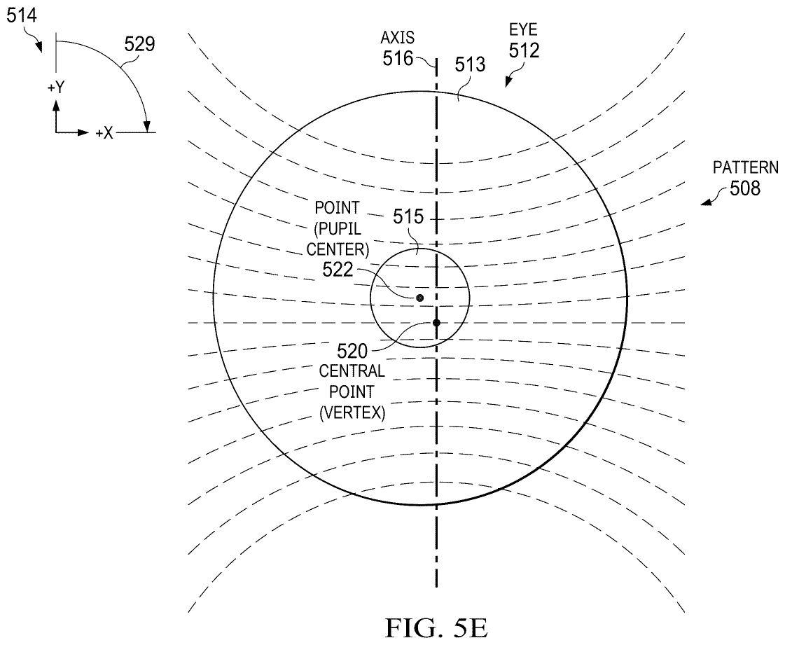

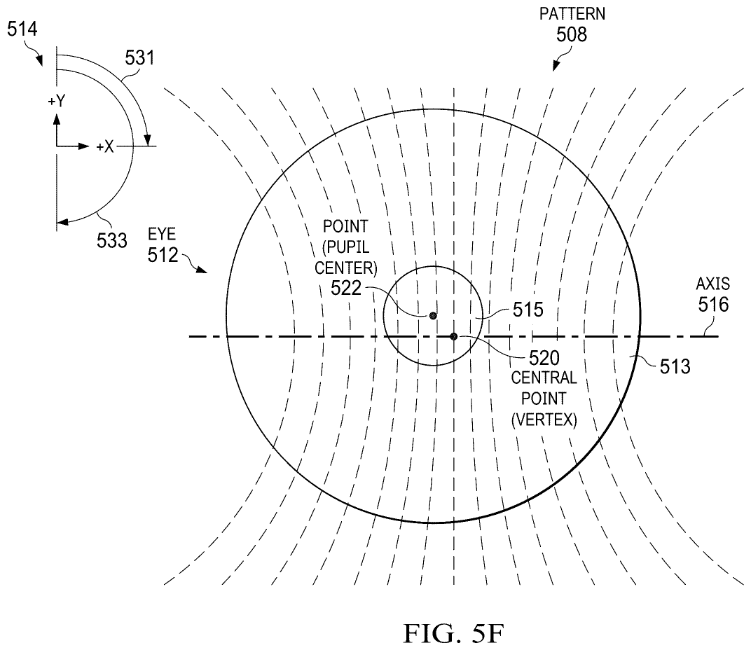

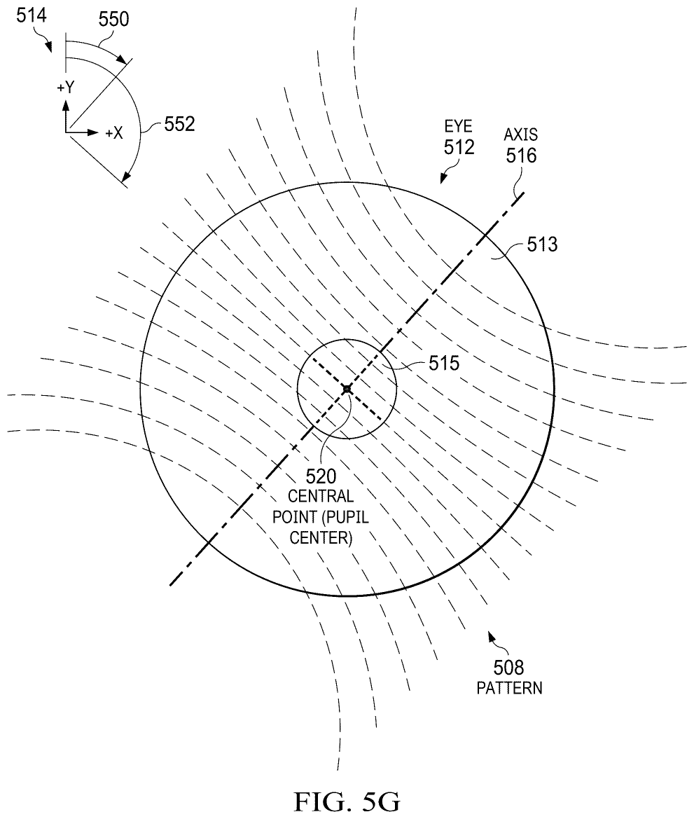

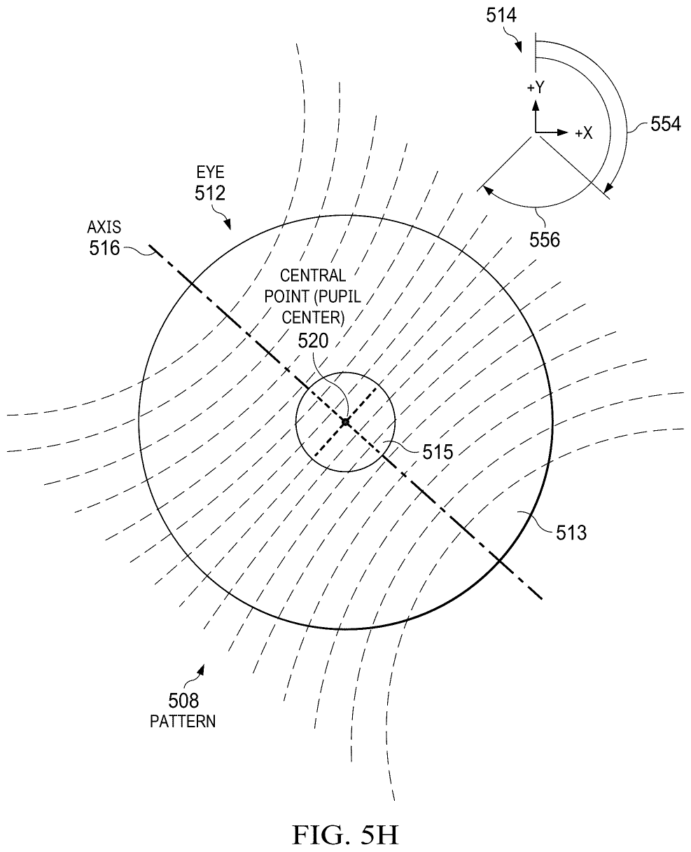

FIGS. 5A to 5H illustrate examples of an alignment pattern 508 directed at different positions of an eye 512, according to at least one embodiment described in the present disclosure. For ease of explanation, a coordinate system 514 with a +x-axis and a +y axis is shown to describe the position (e.g., location and/or orientation) of the alignment pattern 508. The coordinate system 514 may be the coordinate system of, e.g., an ophthalmic system, a laser device, an imaging system, an illuminator, an eye tracker, and/or other device. The alignment pattern 508 has a pattern axis 516. The eye 512 has an iris 513, a pupil 515, and a central point 520. As described herein, the central point 520 of the eye 512 may refer to the corneal vertex, the corneal apex, the pupil center, or other suitable point or may refer to a point defined relative to one of or any combination of the corneal vertex, the corneal apex, the pupil center, or other suitable point.

The alignment pattern 508 may be positioned relative to the coordinate system 514 in any suitable manner. As discussed above, the position of the alignment pattern 508 may indicate the direction of the perpendicular misalignment. In certain embodiments, a system may direct the alignment pattern 508 to different positions in order to determine the direction and/or amount of misalignment. In the embodiments, the system may select the positions of the alignment pattern 508 in any suitable manner. For example, the computer may receive a user selection of the positions of the alignment pattern 508. As another example, the computer may select the positions of the alignment pattern 508 in accordance with the shape of the eye 512, e.g., the shape of the anterior corneal surface of the eye 512. In an example case, if the eye 512 has an oblong, elongated cornea (as with an astigmatic cornea), the pattern axis 516 may be aligned with a meridian of the astigmatism, e.g., with the flatter and/or steeper meridian. As yet another example, the computer may use default positions.

FIGS. 5A and 5B illustrate an example of an alignment pattern 508 directed at different positions of the eye 512. In the example, the alignment pattern 508 is centered about the central point 520 of the eye 512, e.g., the pupil center, and may be rotated about the central point 520 to obtain images of the alignment pattern 508 at different positions.

In FIG. 5A, the pattern axis 516 is parallel to the +y-axis of the coordinate system 514. As discussed above, this position may be used to determine the amount of perpendicular misalignment in a direction parallel to a 90 degree angle 529 from the +y-axis (i.e., parallel to the x-axis).

In FIG. 5B, the pattern axis 516 is parallel to 90 degree angle 531 from the +y-axis (i.e., parallel to the x-axis). This position may be used to determine the amount of perpendicular misalignment in a direction parallel to a 180 degree angle 533 from the +y-axis (i.e., parallel to the y-axis).

FIGS. 5C and 5D illustrate an example of an alignment pattern 508 directed at different positions of the eye 512. In the example, the alignment pattern 508 is centered about the central point 520 of the eye 512, e.g., the pupil center, and may be rotated about the central point 520 to obtain images of the alignment pattern 508 at different positions.

In FIG. 5C, the pattern axis 516 is parallel to a 45 degree angle 530 from the +y-axis. This position may be used to determine the amount of perpendicular misalignment in a direction parallel to a 135 degree angle 532 from the +y-axis.

In FIG. 5D, the pattern axis 516 is parallel to a 135 degree angle 534 from the +y-axis of the coordinate system 514. This position may be used to determine the amount of perpendicular misalignment in a direction parallel to a 225 degree angle 536 from the +y-axis.

FIGS. 5E and 5F illustrate an example of an alignment pattern 508 directed at different positions of the eye 512. In the example, the alignment pattern 508 is centered about the central point 520 of the eye 512, e.g., the corneal vertex, that is distinct from a different point 522 at which the image is centered, e.g., the pupil center. The alignment pattern 508 is rotated about the central point 520 to obtain images of the alignment pattern 508 at different positions.

In FIG. 5E, the pattern axis 516 is parallel to the +y-axis of the coordinate system 514. As discussed above, this position may be used to determine the amount of perpendicular misalignment in a direction parallel to a 90 degree angle 529 from the +y-axis (i.e., parallel to the x-axis).

In FIG. 5F, the pattern axis 516 is parallel to 90 degree angle 531 from the +y-axis (i.e., parallel to the x-axis). This position may be used to determine the amount of perpendicular misalignment in a direction parallel to a 180 degree angle 533 from the +y-axis (i.e., parallel to the y-axis).

FIGS. 5G and 5H illustrate an example of an alignment pattern 508 directed at different positions of the eye 512. In the example, the alignment pattern 508 is centered about the central point 520 of the eye 512 and rotated about the central point 520 to obtain images of the alignment pattern 508 at different positions, where the different positions are selected according to meridians that describe an astigmatism of the eye 112.

In FIG. 5G, the pattern axis 516 is parallel to an angle 550 from the +y-axis that matches a meridian of the astigmatism, such as the steeper meridian. This position may be used to determine the amount of perpendicular misalignment in a direction parallel to an angle 552 that is orthogonal to the angle 550.

In FIG. 5H, the pattern axis 516 is parallel to an angle 554 from the +y-axis that matches a meridian of the astigmatism, such as the flatter meridian. This position may be used to determine the amount of perpendicular misalignment in a direction parallel to an angle 556 that is orthogonal to the angle 554.

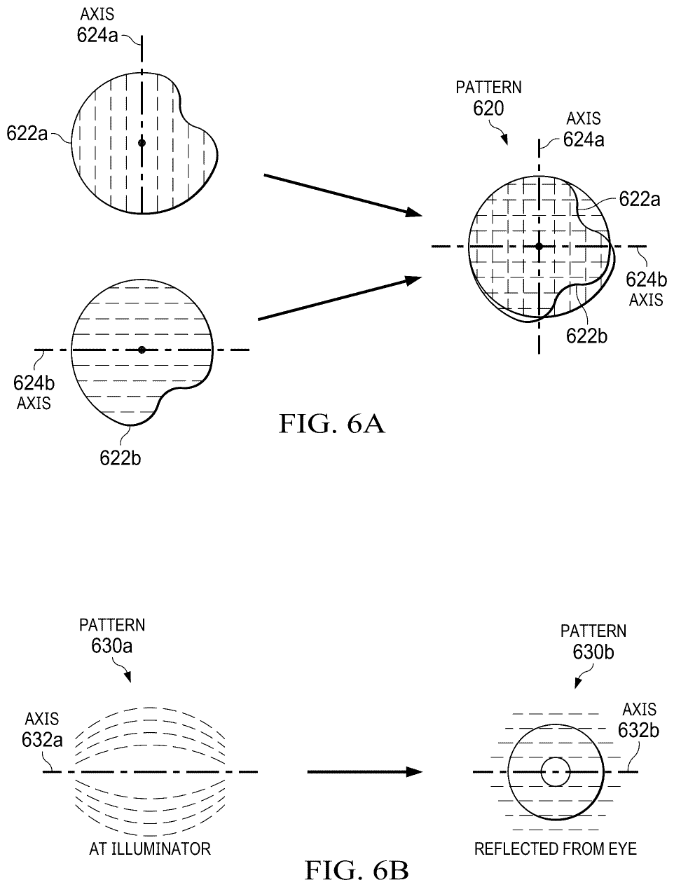

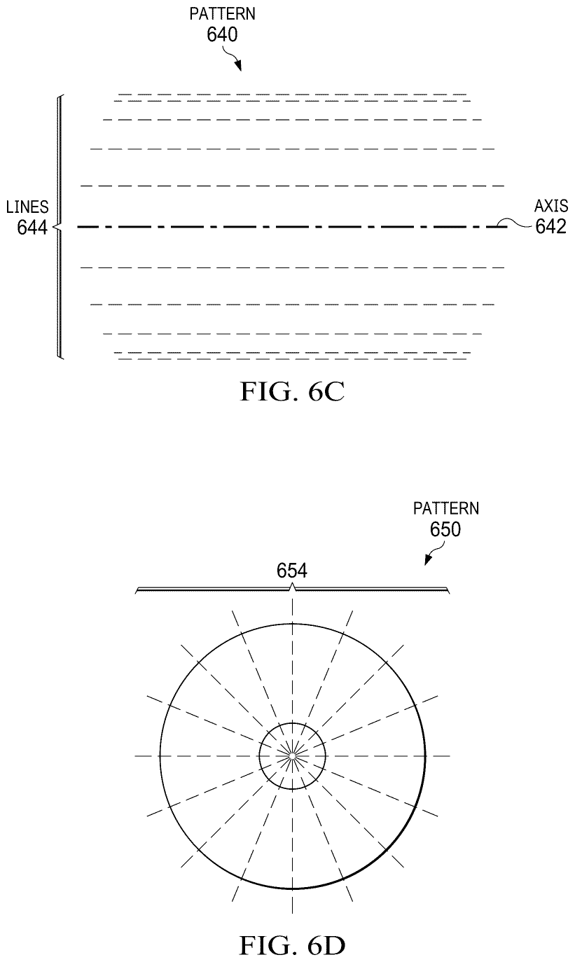

FIGS. 6A to 6D illustrates examples of patterns 620, 630, 640, and/or 650 that can be directed towards an eye, according to at least one embodiment described in the present disclosure. The patterns 620, 630, 640, and/or 650 may be used as an alignment pattern and/or a test pattern. As described herein, a pattern 620, 630, 640, and/or 650 may have any suitable number of any suitable geometric objects arranged in any suitable manner that, when reflected from an eye, indicates the perpendicular alignment of the eye and/or the shape of the anterior corneal surface.

FIG. 6A illustrates an example pattern 620. The pattern 620 includes sub-patterns 622a and 622b with axes 624a and 624b, respectively. The sub-patterns 622a and 622b may be any suitable patterns and may be similar to each other or different from each other. The outlines of the sub-patterns 622a and 622b are drawn only to indicate the position of the sub-patterns 622a and 622b and do not represent the shape of the sub-patterns 622a and 622b.

In an example, the sub-pattern 622b may be substantially similar to the sub-pattern 622a, except that the axis 624b is at an angle of 80 to 100 degrees, e.g., 90 degrees, relative to the axis 624a. For example, the sub-pattern 622a may comprise a number of lines that are perpendicular to the axis 624a, and the sub-pattern 622b may comprise a number of lines that are perpendicular to the axis 624b, e.g., the sub-pattern 622b may comprise lines that are perpendicular to the lines of the sub-pattern 622a. In the example, the resulting pattern 620 may comprise a rectangular grid of lines.

FIG. 6B illustrates an example pattern 630 (630a and 630b) with an axis (632a and 632b, respectively). The pattern 630a is shown at a pattern illuminator, and the pattern 630b is shown as reflected by an eye that is perpendicularly aligned. The pattern 630 is designed such that curved lines of the pattern 630a at the pattern illuminator become straight lines in the pattern 630b as reflected by the perpendicularly aligned eye.

FIG. 6C illustrates an example pattern 640 with an axis 642 and lines 644 that are parallel to the axis 642. The lines may have any suitable curvature, e.g., the lines may be straight at an illuminator and curved when reflected by an eye, or the lines may be curved at an illuminator and straight when reflected by an eye. In the example, the distance between adjacent lines closer to the axis 642 is greater than the distance between adjacent lines farther from the axis 642. This may increase resolution of the determination of perpendicularity towards the periphery of the eye.

FIG. 6D illustrates an example pattern 650. The pattern 650 includes any suitable number of lines 652 that intersect at one or more points, e.g., a central point of an eye. Two or more lines 652 may intersect at the same point, or different lines 652 may intersect at different points.

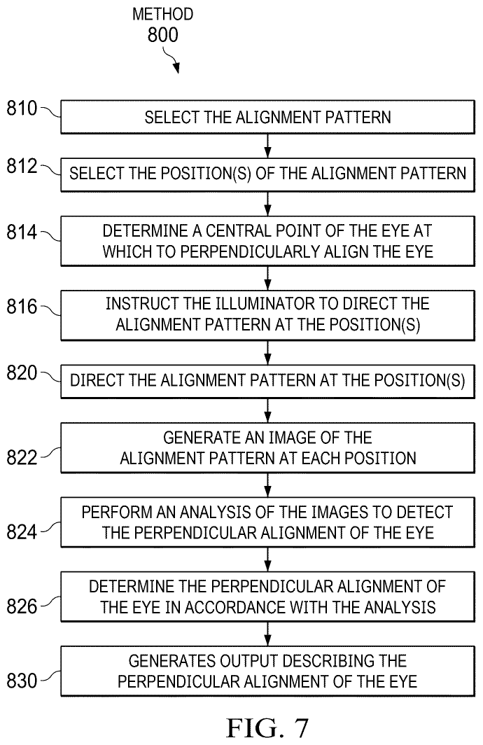

FIG. 7 illustrates an example of a method 800 for determining the perpendicular alignment of an eye, according to at least one embodiment described in the present disclosure. The method may be performed by any suitable system and/or computer system described herein.

At block 810, a computer selects the alignment pattern. The computer may select the alignment pattern in any suitable manner, e.g., according to a default alignment pattern, a user selection, and/or the shape of the anterior surface.

At block 812, the computer selects one or more position(s) at which to direct the alignment pattern. The computer may select the position(s) in any suitable manner, e.g., according to a default position, a user selection, and/or the shape of the anterior surface.

At block 814, a computer determines a central point of the eye with which the system is to be perpendicularly aligned. The computer may determine the central point by accessing a treatment plan indicating the central point and/or receiving user input indicating the central point.

At block 816, the computer instructs an illuminator to direct the alignment pattern. The instructions may designate the alignment pattern, the position(s) of the alignment pattern, and/or the center point of the alignment.

At block 820, the illuminator directs an alignment pattern towards the eye at the position(s). The different position(s) of the alignment pattern may be achieved by moving the illuminator (e.g., rotating the illuminator), adjusting the scanning of the pattern, and/or by turning on/off lights of an array such that the pattern moves to the positions. In certain embodiments, the illumination of the alignment pattern may be coordinated with an eye-tracker. For example, a computer may instruct the illuminator to cease the illumination of the alignment pattern when the eye-tracker is directing an eye-tracking light towards the eye.

At block 822, the camera system generates an image of the alignment pattern reflected from the eye at each position of the position(s) to yield one or more image(s).

At block 824, the computer performs an analysis of the image(s) to detect the perpendicular alignment of the eye with the ophthalmic system.

At block 826, the computer determines the perpendicular alignment of the eye with the ophthalmic system in accordance with the analysis. In certain embodiments, the computer performs the analysis by: detecting the alignment pattern in an image and determining if the alignment pattern has a distortion indicating that there is a misalignment. In the embodiments, the computer may determine the direction and/or the amount of the misalignment.

At block 830, the computer generates output in response to the perpendicular alignment of the eye. For example, the output may be a notification indicating the direction and/or amount of the misalignment, a warning indicating that the misalignment is within a caution zone, a warning indicating that the misalignment exceeds the safe procedure limit, and/or a pause in the operation of a laser.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to” or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a” or “an” or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. § 112(f).

What is claimed:

1. A system for aligning an eye, comprising:
an illuminator configured to:
direct an alignment pattern towards the eye at one or more positions, the alignment pattern designed to indicate a perpendicular alignment of the eye with an ophthalmic system;
a camera system configured to:
generate an image of the alignment pattern reflected from the eye at each position of the one or more positions to yield one or more images; and
a computer configured to:
perform an analysis of the one or more images to detect the perpendicular alignment of the eye with the ophthalmic system; and
determine the perpendicular alignment of the eye with the ophthalmic system in accordance with the analysis.

2. The system of claim 1, the computer configured to:
provide an output representing the perpendicular alignment of the eye with the ophthalmic system.

3. The system of claim 1, the computer configured to perform the analysis of the one or more images to detect the perpendicular alignment of the eye with the ophthalmic system by:
detecting the alignment pattern in the one or more images; and
determining if at least a portion of the alignment pattern has a distortion indicating that there is a misalignment of the eye with the ophthalmic system.

4. The system of claim 3, the computer configured to:
determine, according to the distortion, a direction of the misalignment of the eye.

5. The system of claim 3, the computer configured to:
determine, according to a degree of the distortion, an amount of the misalignment of the eye.

6. The system of claim 3, the computer configured to:
determine a direction of the misalignment of the eye; and
provide an output indicating the direction of the misalignment of the eye.

7. The system of claim 3, the computer configured to:
determine an amount of the misalignment of the eye; and
provide an output indicating the amount of the misalignment of the eye.

8. The system of claim 1, the one or more positions of the alignment pattern comprising:
a first position of the alignment pattern; and
a second position of the alignment pattern, the second position of the alignment pattern rotated about a central point of the eye away from the first position of the alignment pattern.

9. The system of claim 8, the second position of the alignment pattern rotated about the central point of the eye away from the first position of the alignment pattern by an angle with a value between 25 and 95 degrees.

10. The system of claim 1, the illuminator configured to direct the alignment pattern towards the eye at the one or more positions by:
directing a first alignment pattern towards the eye at one or more first positions; and
directing a second alignment pattern towards the eye at one or more second positions.

11. The system of claim 10, the camera system configured to generate the image of the alignment pattern reflected from the eye at the each position of the one or more positions to yield the one or more images by:
generating a first image of the first alignment pattern reflected from the eye at the each first position of the one or more first positions to yield one or more first images; and
generating a second image of the second alignment pattern reflected from the eye at the each second position of the second one or more positions to yield one or more second images.

12. The system of claim 10, the computer configured to perform the analysis of the one or more images to detect the perpendicular alignment of the eye with the ophthalmic system by:
performing a first analysis of one or more first images of the first alignment pattern reflected from the eye to detect the perpendicular alignment of the eye with the ophthalmic system; and
performing a second analysis of one or more second images of the second alignment pattern reflected from the eye to detect the perpendicular alignment of the eye with the ophthalmic system.

13. The system of claim 1, the computer configured to:
determine a central point of the eye with which the ophthalmic system is to be perpendicularly aligned.

14. The system of claim 13, the computer configured to determine the central point of the eye with which the ophthalmic system is to be perpendicularly aligned by:
accessing a treatment plan indicating the central point.

15. The system of claim 13, the computer configured to determine the central point of the eye with which the ophthalmic system is to be perpendicularly aligned by:
receiving a user input indicating the central point.

16. The system of claim 13, the illuminator configured to direct the alignment pattern towards the eye such that the alignment pattern is centered about the central point.

17. The system of claim 1, the illuminator is configured to cease directing the alignment pattern towards the eye when an eye-tracker is directing an eye-tracking light towards the eye.

18. The system of claim 1, the computer configured to:
access a description of a shape of an anterior surface of the eye; and
design the alignment pattern according to the description of the shape of the anterior surface of the eye.

19. The system of claim 1, the computer configured to:
access a description of a shape of an anterior surface of the eye; and
select at least one position of the one or more positions according to the description of the shape of the anterior surface of the eye.

20. The system of claim 1:
the illuminator configured to direct a test pattern towards the eye at one or more test positions;
the camera system configured to generate a test image of the test pattern reflected from the eye at each test position of the one or more test positions to yield one or more test images; and
the computer configured to perform a test analysis of the one or more test images to yield a description of a shape of an anterior surface of the eye.

21. The system of claim 20, the computer configured to:
design the alignment pattern according to the description of the shape of the anterior surface of the eye.

22. The system of claim 20, the computer configured to:
select at least one position of the one or more positions according to the description of the shape of the anterior surface of the eye.

23. The system of claim 1, the computer configured to:
determine that a perpendicular misalignment exceeds a safe procedure limit; and
pause an operation of a laser of the ophthalmic system.

24. The system of claim 1, the computer configured to:
determine that a perpendicular misalignment exceeds a safe procedure limit; and
provide an output indicating that the perpendicular misalignment exceeds the safe procedure limit.

25. The system of claim 1, the computer configured to:
determine that a perpendicular misalignment is within a caution zone; and
provide an output indicating that the perpendicular misalignment is within the caution zone.
